(19) United States
(12) Patent Application Publication
(10) Pub. No.: US 2023/0031763 A1
(43) Pub. Date: Feb. 2, 2023

(54) DEVICE FOR DELIVERING MEDICATION INCLUDING INTERPOSER

(71) Applicant: AITA BIO INC., New york, NY (US)

(72) Inventor: Peter Smeys, San Jose, CA (US)

(21) Appl. No.: 17/783,223

(22) PCT Filed: Dec. 9, 2020
(86) PCT No.: PCT/US20/63959

§ 371 Date: Jun. 7, 2022

Related U.S. Application Data
(60) Provisional application No. 62/946,382, filed on Dec. 10, 2019.

(51) Int. Cl. A61M 5/172; A61M 5/142; A61M 5/145

(52) U.S. Cl. CPC A61M 5/1723; A61M 5/14244; A61M 5/145; A61M 2230/201; A61M 2205/3331; A61M 2005/1726

(57) ABSTRACT

A device is disclosed that is configured as a fully autonomous and integrated wearable apparatus for diabetes management. The device comprises a reservoir for storing the medication for subsequent delivery to a patient, a needle for delivering the medication to the patient subcutaneously, a micropump for pumping the medication from the reservoir through the needle for delivering the medication to the patient, control circuitry controlling operations of the micropump, an interposer configured as an adapter for (1) mounting the reservoir, micropump, the control circuitry and the needle, and (2) distributing medication through one or more channels within the interposer from the reservoir to the needle via the micropump and electrical signals between the control circuitry and micropump.

### REFERENCE TO RELATED APPLICATIONS

This application claims priority to U.S. provisional application No. 62/946,382, filed on Dec. 10, 2019 entitled “Device For Delivering Insulin Including Interposer,” which is incorporated by reference herein.

### FIELD OF THE INVENTION

The present invention relates to a device for delivering insulin including an interposer.

### BACKGROUND OF THE INVENTION

Various infusion systems exist that utilize devices for delivering liquid medication or other therapeutic fluid to patients subcutaneously. For patients with diabetes mellitus, for example, conventional infusion systems incorporate various pumps that are used to deliver insulin to a patient. These pumps have the capability of delivering assorted fluid delivery profiles which include specified basal rates and bolus requirements. For example, these pumps include a reservoir to contain the liquid medication along with electromechanical pumping technology to deliver the liquid medication via tubing to a needle that is inserted subcutaneously into the patient.

Although such conventional pumps/infusion systems are adequate for their intended purpose, such pumps have difficult controlling drug delivery precisely thereby causing harm to the patient. That is, these pumps have large stroke volumes resulting in inaccurate basal rate infusion and incorrect insulin dosing. Further, with these infusion systems, diabetes patients must install and carry at least two bulky and obtrusive devices on their bodies. This causes significant inconvenience for the patient during his/her daily activities.

Therefore, it would be advantageous to provide an improved infusion system over these conventional infusion systems.

### SUMMARY OF THE INVENTION

A device for delivering medication including an interposer is disclosed.

In accordance with an embodiment of the disclosure, an interposer is disclosed that is used in a device that is configured as a fully autonomous and integrated wearable apparatus for diabetes management, the interposer configured as an adapter for (1) mounting a reservoir, a micropump, control circuitry and a needle, and for (2) distributing medication through the interposer from the reservoir to the needle via the micropump and electrical signals between the control circuitry and micropump, the interposer comprising: a first side and a second opposing side and a plurality of ports on the first side and a plurality of ports on the second side; and a plurality of channels connecting the plurality of ports on the first side with the plurality of ports on the second side to enable the plurality of ports on the first side to communicate with the plurality of ports on the second side.

In accordance with another embodiment of the disclosure, a device configured as a fully autonomous and integrated wearable apparatus for diabetes management, the device comprising; reservoir for storing the medication for subsequent delivery to a patient; a needle for delivering the medication to the patient subcutaneously; a micropump for pumping the medication from the reservoir through the needle for delivering the medication to the patient; control circuitry controlling operations of the micropump; and an interposer configured as an adapter for (1) mounting the reservoir, micropump, the control circuitry and the needle, and (2) distributing medication through one or more channels within the interposer from the reservoir to the needle via the micropump and electrical signals between the control circuitry and micropump.

In accordance with another embodiment of this disclosure, a device configured as a fully autonomous and integrated wearable apparatus for diabetes management, the device comprising; a reservoir for storing the medication for subsequent delivery to a patient; a needle for delivering the medication to the patient subcutaneously; one or more MEMS devices configured as a micropump for functioning as a pump for pumping the medication from the reservoir through the needle for delivering the medication to the patient or as a microvalve for functioning as a valve for preventing medication from flowing through device; control circuitry controlling operations of the micropump; and an interposer configured as an adapter for (1) mounting the reservoir, micropump, the control circuitry and the needle, and (2) distributing medication through one or more channels within the interposer from the reservoir to the needle via the micropump and electrical signals between the control circuitry and micropump.

### BRIEF DESCRIPTION OF DRAWINGS

FIG. 1 depicts a block diagram of the components of an example device for delivering insulin to a diabetes patient.

FIG. 2 depicts a top view of an example interposer shown in FIG. 1.

FIG. 3 depicts a cross sectional view of the example interposer in FIG. 2 along lines 3-3.

FIG. 4 depicts a perspective view of the assembly of certain components of the device in FIG. 1.

FIG. 5A depicts another perspective view of the assembly of components of the device in FIG. 1.

FIG. 5B depicts an exploded view of the assembly of components of the device in FIG. 1.

FIGS. 6-8 depict various embodiments of a device for delivering insulin to a diabetes patient.

### DETAILED DESCRIPTION OF THE INVENTION

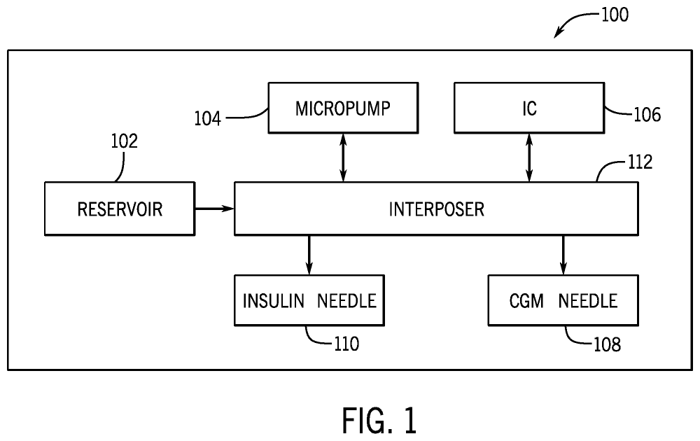

FIG. 1 depicts a block diagram of the salient components of device 100 for delivering insulin (or other medication or fluid) to a diabetes patient (also referred to as a user of device 100). Device 100 is configured as a fully autonomous and integrated wearable apparatus for diabetes management in which continuous glucose monitoring (CGM), insulin delivery and control functionality are provided together to ensure insulin is delivered at very precise rates. Device 100 includes several components or modules including, among other components, reservoir 102, micropump 104 (also referred to as small pump or pump), control circuitry (integrated circuit—IC) 106, insulin needle 108, continuous glucose monitoring (GCM) or analyte sensing needle 110 and interposer 112. Device 100 also includes a battery (not shown) that provides power to IC 106 and micropump 104 (battery may be on a printed circuit board (PCB) as described below). Micropump 104 includes one or more MEMS devices (micro-electro-mechanical systems devices, i.e., piezoelectric transducer), as known to those skilled in the art, into its architecture for pump and/or valve functionality. (MEMS device with valve functionality may also be referred to as a microvalve or valve). However, micropump 104 may incorporate other pumping mechanisms to achieve desired results as known to those skilled in the art. (Besides inulin, device 100 may be configured to infuse other medications such as small molecule pharmaceutical solutions, large molecule or protein drug solutions, saline solutions, blood or other fluids known to those skilled in the art.)

Reservoir 102 is configured to store insulin for subsequent delivery to the patient via insulin needle 108 as known to those skilled in the art. Micropump 104 is configured to pump insulin through insulin needle 108 into the patient. Control circuitry 106, as known to those skilled, in the art is configured to control the operation of the micropump 104. CGM or analyte sensor needle 110 is configured to monitor glucose levels in the patients and transmit the data to control circuitry 106.

Interposer 112 is configured as an adapter for (1) mounting reservoir 102, micropump 104, control circuitry (integrated circuit—IC) 106, insulin needle 108 and CGM or analyte sensor needle 110 and for (2) redistributing fluid through channels and electrical signals between those components. That is, interposer 112 functions to fully integrate reservoir 102, micropump 104, control circuitry (IC) 106, insulin needle 108 and GCM or analyte sensor needle 110 in order to reduce the amount of tubing and wiring to connect such components and miniaturize the delivery device. Interposer 112 also includes a flow sensor or pressure sensor (not shown), e.g., as a separate MEMS device, to monitor flowrate of the insulin and/or occlusion of the pump as known to those skilled the art. Interposer 112 is constructed of glass, but it may be made of other materials known to those skilled in the art. With interposer 112, the only connections or openings to the environment is a fill port, needle opening, sensor needle opening and connector pins for connecting power, ground and control signals to a printed circuit board (PCB). This is described in more detail below.

These components of device 100 are mounted to interposer 112 using laser, adhesive bonding, flip chip or other methods known to those skilled in the art. Electrical connections from micropump 104 to interposer 112 are made using wirebond or other means of connection known to those skilled in the art. Electronic connections from IC 106 to interposer 112 are constructed using wirebond, flip chip or other means known to those skilled in the art. Micropump 104 and integrated circuit (IC) 106 may be mounted at wafer level by die to wafer automated pick and place. Reservoir 102 and a spacer 114 (discussed below) may be mounted at wafer level or using other assembly processes known to those skilled in the art. Interposer fabrication is discussed below.

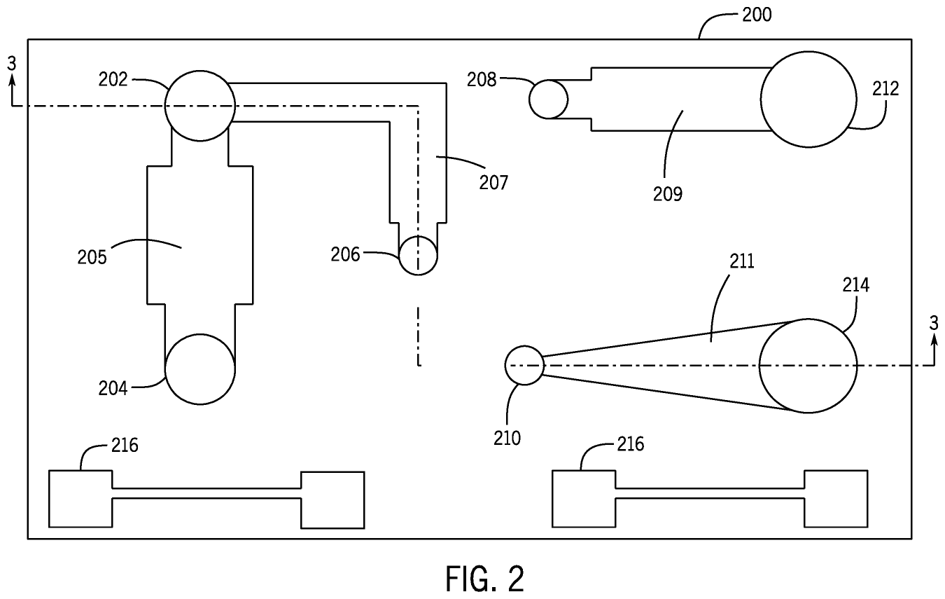

FIG. 2 depicts a top view of interposer 200 as identified in FIG. 1 (reference renumbered). In this figure, fluid channels, inlet and outlet ports and interconnects as shown as described below.

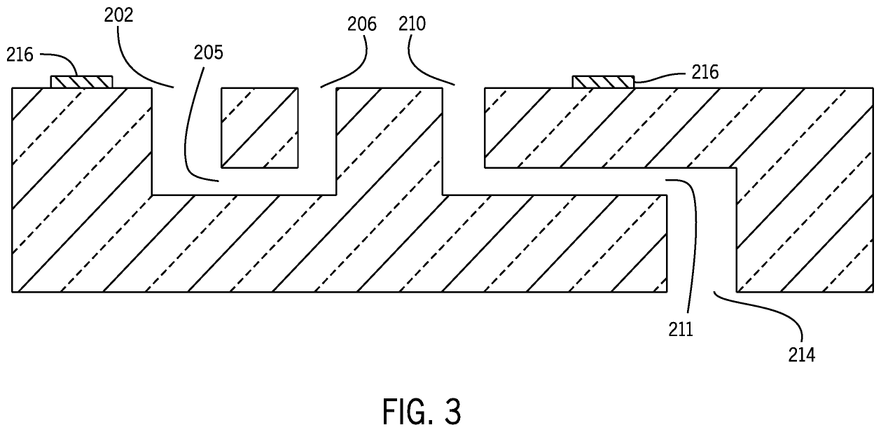

FIG. 3 depicts a cross sectional view of the interposer 200 in FIG. 2 along lines 3-3. This interposer is an example of an adapter that depicts (1) several ports that function as openings for fluid paths (e.g., channels) through the interposer for fluidly connecting reservoir 102, micropump 104, CGM needle 108 and insulin needle 110 and (2) interconnects for electrically connecting components IC 106, micropump 104 and CGM needle 108.

Specifically, interposer 200 includes top ports 202, 204 that are connected by channel 205 as shown as well as top port 206 that communicates with top port 202 via channel 207. Top port 202 may for example communicate with a port on micropump 104 and port 206 may communicate with reservoir 102. Top port 208 communicates with bottom port 212 via channel 209 and port 210 communicate with bottom port and 214 via channel 211. Top ports 208 and 210 may for example communicate with ports on micropump 104 and bottom ports 212 and 214 may communicate with insulin needle 106 and CGM or analyte sensor needle 108, respectfully (or cannulas).

Example measurement for top port 202 (of channel) may be 300 μm, top port 206 (channel) may be 100 μm and top port 210 may be 100-200 μm. Bottom port 212 may be 300 μm. The channel between top port 210 and bottom port 214 may vary but an example may be 25 μm. The height of the interposer 200 may be 800 μm. However, those skilled in the art know that the ports may be configured to various sizes/measurements to achieve desired effects. Interconnects 216 are also shown along with the ports in interposer 200. Interconnects 216 are configured as electrical connectors or conduits that enable the connection between micropump 104, CGM or analyte sensor 108 (and battery) and IC 106 as known to those skilled in the art. Interconnects may have thickness as 20 μm in narrow portion 90 μm as the ends. However, the interconnects may be any measurement to achieve desired results. In short, interposer 200 includes both ports/channels and interconnects to distribute fluid channels and electrical signals, respectively. (Interposer 200 may be constructed of a transparent material such as glass or any other transparent or non-transparent material known to those skilled in the art.)

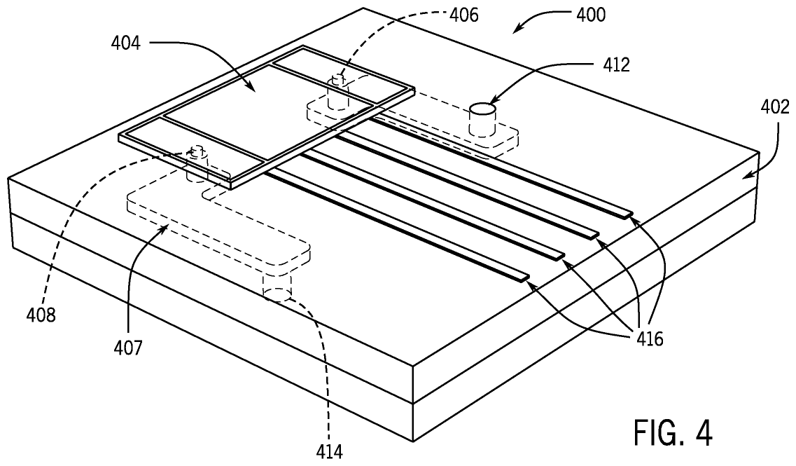

FIG. 4 depicts a perspective view of the assembly of certain components (including micropump) of an example device 400 for delivering insulin to a diabetes patient in FIG. 1 (reference renumbered). That is, FIG. 4 shows an assembly of components such as a micropump onto an interposer as described below.

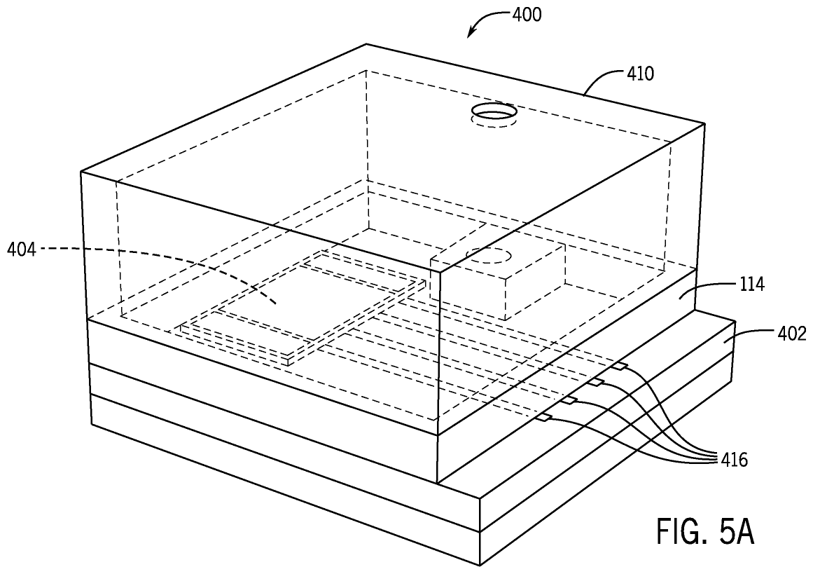

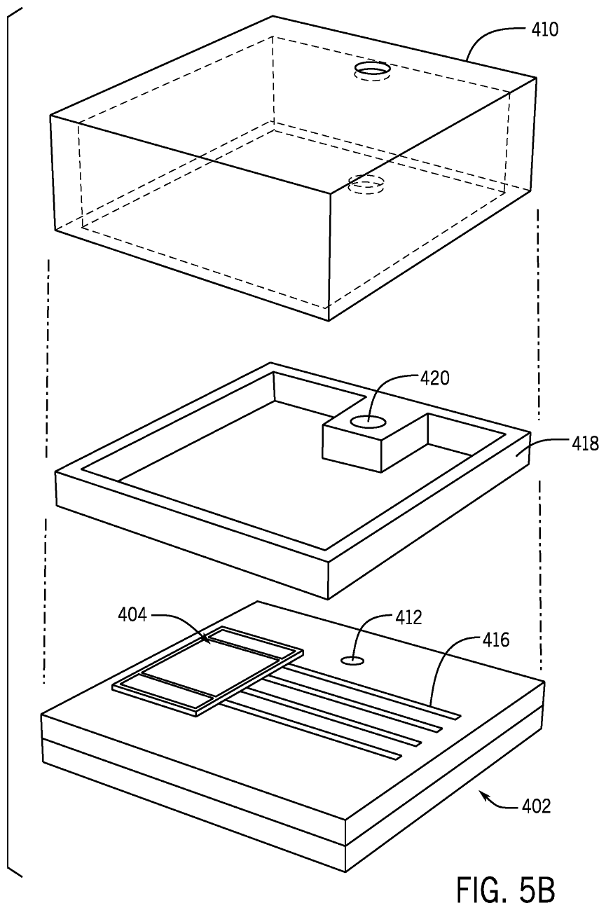

FIGS. 5A and 5B depict perspective views of the assembly of the example device 400 shown in FIG. 1. Example device 400 includes another example interposer 402 (different embodiment than interposer 200 in FIGS. 2 and 3). Device 400 includes micropump 404 (e.g., one or more MEMS devices) that is assembled on top of interposer 402 as shown and it communicates with ports 406, 408 to (1) withdraw insulin from reservoir 410 through inlet port 412 and (2) propel insulin through fluid channel 407 and out outlet port 414. As indicated above, (metal) interconnects 416 are used as connectors (i.e., traces) for connecting micropump 404 to an integrated circuit (IC), battery (not shown in FIGS. 4, 5A, 5B) as known to those skilled in the art. Device 400 also includes spacer 418 between reservoir 410 and interposer 402. Spacer 418 creates a standoff between reservoir 410 and interposer 402 to provide space for the micropump 404 and IC (not shown). Spacer 418 is configured to be of smaller size than interposer 402 to enable external connections as shown. Spacer 418 can be fabricated out of silicon, plastic or other material known to those skilled in the art. Spacer 418 has a through channel 420 that connects interposer 402 to reservoir 410 for drug delivery. That is, channel 420 is configured to enable flow from reservoir 410 into inlet port 412. (Interposer 402 may be constructed as a transparent material such as glass/Silicon or any other transparent or non-transparent material as known to those skilled in the art.)

The process for assembling the components onto an interposer is now described. The process proceeds to step 1, wherein a pump die is mounted onto the interposer using adhesive bonding (die-die or die wafer). (Steps now shown.) Next at step 2, control ICs are mounted onto the interposer using adhesive bonding (die-die or die-wafer). The process proceeds to step 3, wherein the pump and ICs are wirebonded down to the interposer. Next, a spacer is mounted onto the interposer using adhesive bonding at step 4. As indicated above, the spacer creates a standoff between a reservoir and interposer to provide space for the micropump and ICs. As indicated above, the spacer can be fabricated out of silicon, plastic or other material known to those skilled in the art. The spacer has a through hole that connects the interposer to the reservoir for drug delivery. The process proceeds to step 5, wherein the reservoir is mounted onto the spacer using adhesive bonding. The reservoir and spacer can be combined into a single component if desired.

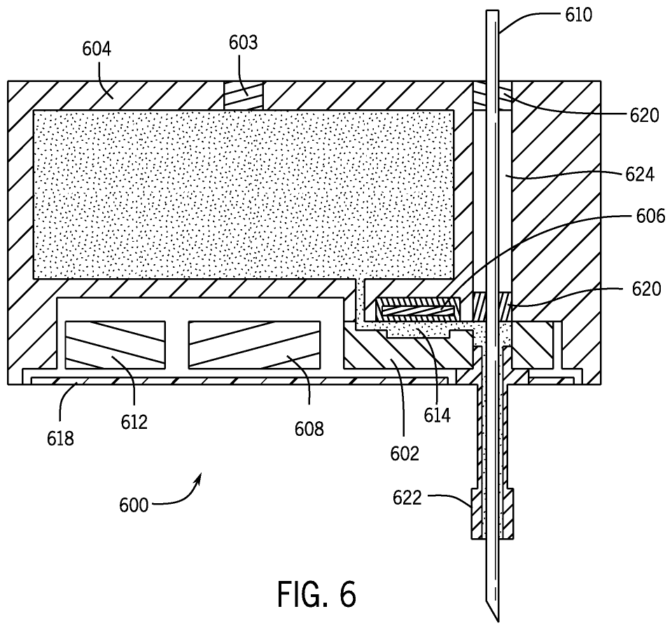

FIGS. 6-8 depict various examples of a device for delivering insulin to a patient. Details appear below.

In FIG. 6, device 600 for delivering insulin to a diabetes patient includes interposer 602 that is configured as an adapter as described hereinabove. Device 600 further includes reservoir 604, micropump 606, control circuitry 608 (ASIC/SOC), insulin needle 610, battery 612 and GCM or analyte sensor needle (not shown) as well as a printed circuit board (PCB) 618. PCB 618 is configured to mount (and electronically connect) micropump 606, control circuitry 608 and battery 612 as well as reservoir 604 as known to those skilled in the art. Battery 612 provides power to control circuitry 608 and micropump 606 on a printed circuit board 616 as known to those skilled in the art. Reservoir 602 is configured above most device 600 components in this example device. Reservoir 602 has a fill port 603 on the top as shown. Micropump 606 drives insulin from reservoir 604 through channel 614 to supply insulin to a cannula as described below. Example measurements for the length of interposer 660 from edge to channel 624 (below) may be 20-30mm but those skilled in the art know that other measurements may be used to achieve desired results.

Device 600 also includes a cannula delivery mechanism that includes silicone stoppers 620, cannula 622 (and needle 610) and channel 624 that extends through most of device 600 as shown. Reservoir 602 acts as a side wall to support a cannula delivery mechanism that houses needle 610. Silicone stoppers 620 function to close off channel 624 in deployed configuration (on patient). Needle 610 is advanced through channel 624 as the stoppers 620 elastically spread/expand to enable needle 610 to translate through channel 624 and out cannula 622 to puncture the skin of the patient. (Needle 610 may be inserted during manufacture.) Cannula 622 is also configured to advance into the patient to enable insulin dosing. Needle 610 is then retracted, leaving the cannula 622 fully deployed in the patient (user). Insulin from channel 614 may then flow as needed.

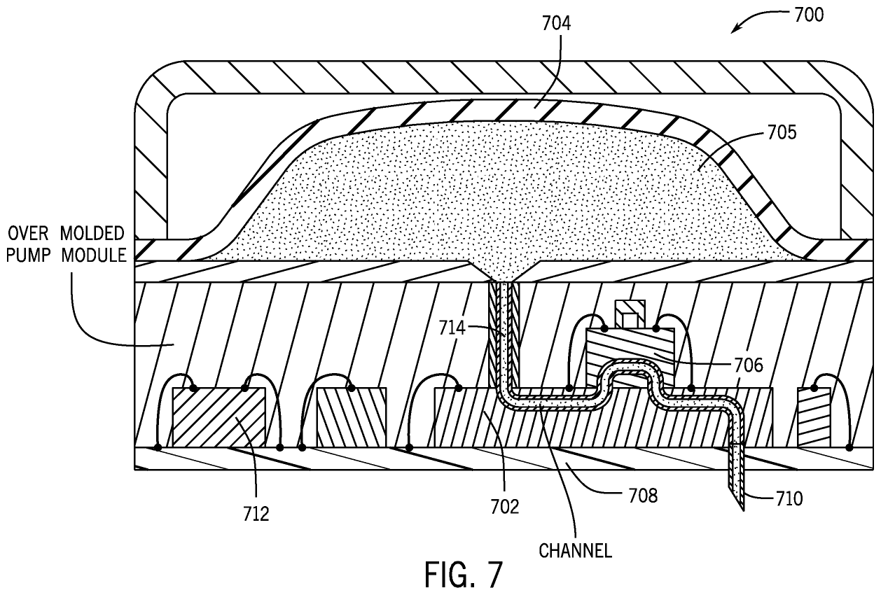

In FIG. 7, device 700 for delivering insulin to a diabetes patient includes interposer 702 that is configured as an adapter as described hereinabove. In this example, device 700 includes reservoir 704 that is defined by a silicone membrane that functions as a silicone bubble or balloon that adjusts to expand or contract as insulin 705 increases and decreases within reservoir 704 and provides a positive pressure to enable direction independent emptying. Device 700 also includes micropump 706, control circuitry (printed circuit board—PCB) 708, insulin needle 710, CGM or analyte sensor needle (not shown) and battery 712. Similar to the example in FIG. 6, PCB 712 is configured to mount and electronically connect many of the components of device 700 (including reservoir 704 on top). Specifically, micropump 606, control circuitry 708 and battery 712 are mounted directly to the PCB 712 as shown.

Needle 710 also functions as a channel that leads or extends through sections of interposer 702 (i.e., interposer channel) as well as into and through micropump 706. Device 700 includes a channel 714 between reservoir 704 and interposer 702 that communicates with this interposer channel within interposer 702 that is defined as the needle 710 as described above (and ultimately micropump 706). Micropump 706 functions to draw insulin from reservoir 704 through channel 714 channel and propel insulin through micropump 706 and out needle 710 as known to those skilled in the art. In this example, reservoir 704 is configured with a silicone membrane that functions as a silicone bubble or balloon that adjusts to insulin increase and decrease changes within reservoir 704 and provides a positive pressure to enable direction independent emptying or discharging. In this example, needle 710 remains within the patient. In assembly, several components are mounted directly to PCB 708 including interposer 702. Then, plastic package film assisted molding is used to encapsulate all the components and create the connection to the reservoir. Next, a reservoir section is mounted to the resulting section to create a device 700.

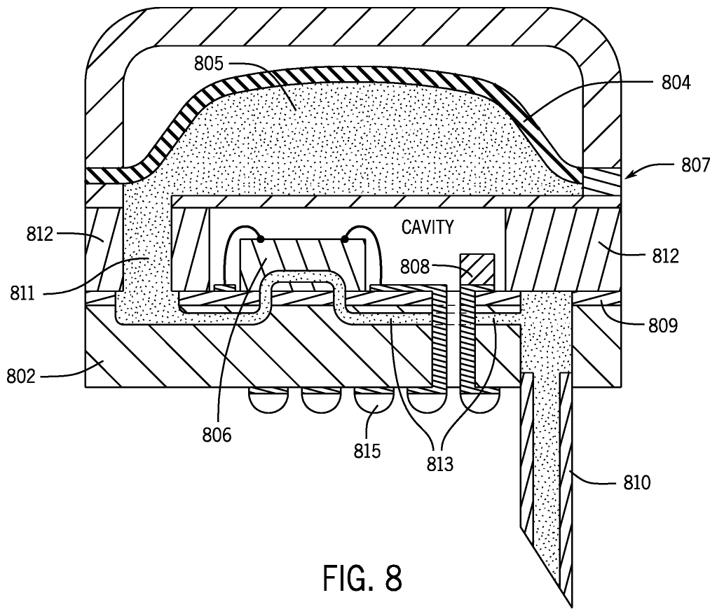

In FIG. 8, example device 800 for delivering insulin to a patient is shown which includes interposer 802 that is configured as an adapter as described hereinabove. In this example, device 800 also includes reservoir 804 that is defined by a silicone membrane that functions to create a silicone bubble or balloon that adjusts to changes in insulin 805 quantity within reservoir 804. Fill port 807 appears on the side of device 800 to enable reservoir to be filled with insulin. Device 800 also includes micropump 806, control circuitry (IC) 808, insulin needle 810, GCM or analyte sensor needle and battery (both not shown) and spacer 812 (functions as described above). Reservoir 804 communicates with needle 810 through the channels 811, 813 within spacer 812, interposer 802 and micropump 806 as shown. Interposer 802 is a ball grid array (BGA) 815 interposer that is soldered onto the PCB 809 directly with metal filled through glass vias (TGVs) for flip chip mounting of the device 800 onto the system PCB 809. In this example needle 810 remains within the patient upon deployment.

The example interposers described herein may be fabricated as follows.

Mask 1: Pattern thru-holes in 400 μm thick glass wafer. A through hole size is 300 μm (e.g.).

Sputter 1 μm (e.g.) Aluminum (Al) on a 2^{nd} glass wafer.

Mask 2: Pattern and etch an Al interconnect.

Mask 3: Pattern the backside channels in a glass wafer.

Mask 4: Pattern vias (i.e., holes) in the glass wafer.

Bond glass wafers together.

The process above are examples steps to fabricate a glass interposer. Those skilled in the art know the process steps above may be modified or order changed to achieve desired results (with glass or other material).

It is to be understood that the disclosure teaches examples of the illustrative embodiments and that many variations of the invention can easily be devised by those skilled in the art after reading this disclosure and that the scope of the present invention is to be determined by the claims below.

1. A device configured as a fully autonomous and integrated wearable apparatus for diabetes management, the device comprising:
a reservoir for storing the medication for subsequent delivery to a patient;
a needle for delivering the medication to the patient subcutaneously;
a micropump for pumping the medication from the reservoir through the needle for delivering the medication to the patient;
control circuitry controlling operations of the micropump; and
an interposer configured as an adapter for (1) mounting the reservoir, micropump, the control circuitry and the needle, and (2) distributing medication through one or more channels within the interposer from the reservoir to the needle via the micropump and electrical signals between the control circuitry and micropump.

2. The device of claim 1 wherein the interposer has one or more ports on a first side of the interposer and one or more ports on a second side of the interposer, the one or more ports on the first side communicate with the one or more ports on the second side via the one or more channels.

3. The device of claim 1 wherein the micropump includes a channel that communicates with the one or more channels so that the medication is distributed from the reservoir to the needle through the micropump.

4. The device of claim 1 wherein the micropump includes one or more MEMS devices with pump and/or valve functionality.

5. The device of claim 1 further comprising a flow sensor or pressure sensor to monitor flowrate of the medication and/or occlusion of the micropump.

6. The device of claim 4 5 wherein the flow sensor or pressure sensor is a separate MEMS device.

7. The device of claim 1 further including a continuous glucose monitoring needle configured to monitor glucose levels in the patient and transmit data to the control circuitry.

8. The device of claim 7 further comprising a plurality of interconnects electrically connecting the control circuity, micropump and continuous glucose monitoring needle.

9. The device of claim 1 wherein the medication is insulin.

10. The device of claim 8 wherein the plurality of interconnects transmit the electrical signals from between the control circuitry and micropump.

11. The device of claim 1 further comprising a battery for providing power to the control circuitry and micropump.

12. The device of claim 1 further comprising
first and second ports on first and second sides of the device and a cannula delivery mechanism including:
a channel extending through the device from the first port to the second port, the channel communicates with the one or more channels of the interposer;
a cannula extending from the second side that communicates with the second port and the channel; and
one or more stoppers that function to close off the channel to prevent medication from traveling through the channel when the cannula is in a deployed configuration on the patient;
wherein the needle is configured (1) to advance through the channel and the one or more stoppers to enable the needle to translate out the cannula and thereby puncture the skin of the patient and (2) to retract through the channel leaving the cannula; and
wherein the cannula is configured to advance into the patient as the needle punctures the skin of the patient to enable medication delivery.

13. An interposer used in a device that is configured as a fully autonomous and integrated wearable apparatus for diabetes management, the interposer configured as an adapter for (1) mounting a reservoir, a micropump, control circuitry and a needle, and for (2) distributing medication through the interposer from the reservoir to the needle via the micropump and electrical signals between the control circuitry and micropump, the interposer comprising:
a first side and a second opposing side and a plurality of ports on the first side and a plurality of ports on the second side; and
a plurality of channels connecting the plurality of ports on the first side with the plurality of ports on the second side to enable the plurality of ports on the first side to communicate with the plurality of ports on the second side.

14. The interposer of claim 13 wherein the device includes a continuous glucose monitoring needle configured to monitor glucose levels in the patient and transmit data to the control circuitry.

15. The interposer of claim 13 wherein the device includes a plurality of interconnects electrically connecting the control circuity, micropump and continuous glucose monitoring needle.

16. The interposer of claim 13 wherein the micropump includes one or more MEMS device.

17. A device configured as a fully autonomous and integrated wearable apparatus for diabetes management, the device comprising:
a reservoir for storing the medication for subsequent delivery to a patient;
a needle for delivering the medication to the patient subcutaneously;
one or more MEMS devices configured as a micropump for functioning as a pump for pumping the medication from the reservoir through the needle for delivering the medication to the patient or as a microvalve for functioning as a valve for preventing medication from flowing through device;
control circuitry controlling operations of the micropump; and
an interposer configured as an adapter for (1) mounting the reservoir, micropump, the control circuitry and the needle, and (2) distributing medication through one or more channels within the interposer from the reservoir to the needle via the micropump and electrical signals between the control circuitry and micropump.
